(19) United States
(12) Patent Application Publication
(10) Pub. No.: US 2005/0214874 A1
(43) Pub. Date: Sep. 29, 2005

(54) Diagnosis and treatment of early pre-Type-1 diabetes utilizing glial fibrillary acidic protein

(75) Inventors: George Jackowski, Kettleby (CA); Xiaomao Li, Toronto (CA)

Correspondence Address: MCHALE & SLAVIN, P.A., 2855 PGA BLVD, PALM BEACH GARDENS, FL, 33410, US

(73) Assignee: Syn X Pharma, Inc., Toronto (CA)

(21) Appl. No.: 11/132,975

(22) Filed: May 18, 2005

Related U.S. Application Data
(62) Division of application No. 09/954,972, filed on Sep. 17, 2001.

(51) Int. Cl. G01N 33/53; G01N 33/567

(52) U.S. Cl. 435/7.2

(57) ABSTRACT

This invention relates to the treatment and diagnosis of Type-1 Diabetes (T1D); particularly to the use of glial fibrillary acidic protein (GFAP) as a mediator of the disease; and most particularly to GFAP binding proteins useful for prediabetes screening and/or staging.

### CROSS REFERENCE TO RELATED APPLICATION

This application is a divisional application of application Ser. No. 09/954,972 filed on Sep. 17, 2001, the contents of which is herein incorporated by reference.

### FIELD OF THE INVENTION

This invention relates to autoimmune (Type 1A) diabetes mellitus (T1D). Specifically, the invention relates to the early diagnosis of pre-Type-1 diabetes based on the discovery that Schwann cell proteins, in particular glial fibrillary acidic protein (GFAP) play a role in early stage autoimmunity, particularly serving as a marker of this process; and most particularly serving for the detection of GFAP binding proteins as the earliest harbingers of future disease risk and providing an unexpected, new target for intervention treatments.

### BACKGROUND OF THE INVENTION

T1D in humans and its premier animal model, the non-obese diabetic (NOD) mouse, are polygenic autoimmune diseases whose penetrance is under control of environmental factors (M. Knip, H. K. Akerblom, Exp Clin Endocrinol Diabetes 107, S93-100 (1999); D. B. Schranz, A. Lernmark, Diabetes Metab Rev 14, 3-29 (1998); G. T. Nepom, W. W. Kwok, Diabetes 47, 1177-84 (1998); J. A. Todd, Pathol Biol (Paris) 45, 219-27 (1997); M. A. McAleer et al., Diabetes 44, 1186-1195 (1995)). Insulin deficiency is the end result of a slowly progressive process, prediabetes, characterized by the accumulation of more and more dense T cell infiltrates around (‘peri-insulitis’) and eventually inside the islet (‘invasive insulitis’).

This slow progression and its biological controls are not well understood. Without ready access to the sparsely distributed islets in the human pancreas, most concepts of prediabetes progression derive from the rodent models of the disease (A. A. Rossini, E. S. Handler, J. P. Mordes, D. L. Greiner, Clin Immunol Immunopathol 74, 2-9 (1995); M. A. Atkinson, E. H. Leiter, Nat Med 5, 601-4 (1999)). However, there is strong concensus that human T1D is also characterized by the development of T cells and autoantibodies that recognize β-cell constituents, the former are effectors of β-cell demise during a decade or more of clinically silent prediabetes.

Early NOD prediabetes has successfully been targeted by multiple immunotherapies that slow or altogether halt its progression to overt insulin deficiency and thus diabetes (M. A. Atkinson, E. H. Leiter, Nat Med 5, 601-4 (1999); S. Winer et al., J Immunol 165, 4086-4094 (2000); D. L. Kaufman et al., Nature 366, 69-72 (1993); R. Tisch et al., Nature 366, 72-75 (1993); J. Tian et al., Nature Med. 2, 1348-1353 (1996); J. Tian et al., J Exp Med 183, 1561-7 (1996); J. Tian, C. Chau, D. L. Kaufman, Diabetologia 41, 237-40 (1998); R. Tisch, R. S. Liblau, X. D. Yang, P. Liblau, H. O. McDevitt, Diabetes 47, 894-9 (1998); R. Tisch et al., J Immunol 166, 2122-2132 (2001); J. F. Elliott et al., Diabetes 43, 1494-1499 (1994)). These immunotherapies have all targeted specific autoimmune responses as measured by autoantibodies. The therapeutic effects of the particular autoantigens or relevant epitope peptide fragments from these molecules, derive from the route of application (usually systemically rather than locally), with mechanisms of prediabetes delay or cessation ascribed to clonal deletion, anergy induction and modifications of disease-associated cytokine bias. Unfortunately, the autoantibody responses targeted by these immunotherapies appear relatively late in prediabetes (R. B. Lipton et al., Amer J Epidemiol 136, 503-12 (1992); R. B. Lipton et al., Diabet Med 9, 224-32 (1992)). Treatments are effective only if applied earlier in prediabetes, while later treatments can precipitate overt disease (K. Bellmann, H. Kolb, S. Rastegar, P. Jee, F. W. Scott, Diabetologia 41, 844-847 (1998); R. Tisch, B. Wang, D. V. Serreze, J Immunol 163, 1178-1187 (1999); S. Winer et al., J Immunol 165, 4086-4094 (2000)).

Nevertheless, these observations have engendered optimism in the field that organ-selective autoimmune diseases such as T1D can be successfully prevented in humans at risk for the disease, by immunological interventions that modify the progression of early disease stages. In this, the pressing need for earlier diagnosis of diabetes risk is clear. The present invention represents by far the earliest T1D risk marker identified, and it entails a new therapeutic strategy for early intervention therapy.

In the United States, these developments and needs have been acknowledged by considerable increases in funding for diabetes research, including the development of NIH-sponsored, $300 million research efforts such as THE IMMUNE TOLERANCE NETWORK, TRIGR and TRIALNET. These efforts are aimed at unifying strategies for the translation of animal data to human clinical intervention/prevention trials in organ-selective autoimmune diseases, with T1D the leading concern-reflecting its 100+billion dollar annual cost in the US (˜80% of the total diabetes burden).

The past two decades of human T1D research had as its main theme the development of techniques that would allow reliable detection of prodromal disease states and prediabetes (W. Karges, et al., Molec Aspects Med 16, 79-213 (1995); D. B. Schranz, A. Lernmark, Diabetes Metab Rev 14, 3-29 (1998); R. B. Lipton et al., Amer J Epidemiol 136, 503-12 (1992); R. B. Lipton et al., Diabet Med 9, 224-32 (1992); C. F. Verge et al., Diabetes 45, 926-33 (1996); W. Woo et al., J Immunol Methods 244, 91-103. (2000)).

International workshops continue to provide important controls and improvements in these diagnostic efforts (C. F. Verge et al., Diabetes 47, 1857-66 (1998); R. S. Schmidli, P. G. Colman, E. Bonifacio, and Participating Laboratories, Diabetes 44, 631-635 (1995); R. S. Schmidli, P. G. Colman, E. Bonifacio, G. F. Bottazzo, L. C. Harrison, Diabetes 43, 1005-9 (1994); N. K. MacLaren, K. Lafferty, Diabetes 42, 1099-1104 (1993)). However, while the accuracy of pre-diabetes diagnostics is now approching 90%, it is clear that present autoimmune serology detects only the mid- to late stages of the process with confidence. These stages are characterized in animal models as largely resistant to intervention, and immunotherapy at these stages can accelerate progression and precipitate overt disease (reviewed in S. Winer et al., J Immunol 165, 4086-4094 (2000)).

Thus, the need for very early detection of T1D-risk and impending prediabetes is pressing. While most current studies focus on families with the disease, such techniques must eventually be applicable to the general population, since 85% of new patients do not have a family history of autoimmune disease (W. Karges, J. Ilonen, B. H. Robinson, H.-M. Dosch, Molec Aspects Med 16, 79-213 (1995).

It is clear that if a marker indicative of the earliest stages of pre-diabetes could be targeted, that a better understanding and staging of early prediabetes would be realized, and that therapeutic strategies and avenues capable of altering the course, progression and/or manifestation of the disease would be realized. Such a marker of early prediabetes is of paramount importance and is probably a prerequisite for successful human intervention trials.

### SUMMARY OF THE INVENTION

The above conclusion has re-kindled intense studies of prodromal autoimmunity in animal models. Recent studies by Toronto researchers have added a new concept in these efforts. Thus Winer et al. reported that T1D and multiple sclerosis (MS) share a near identical set of autoreactivities, including islet reactive T cells in MS and nervous system autoreactivity in T1D (J Immunol 166, 2832-2841, ibid 4751-4756 (2001)). SYN-X Pharma, Inc. of Mississauga, Ontario has developed proteomics approaches to nervous system diseases including MS, with the discovery of new biomarker molecules for these disease processes through the use of modern mass spectrometry instrumentation. This technology was used to search for disease markers common to both diseases. In this ongoing process, SYN-X scientists discovered a diabetes-associated 150 kD molecule that reacted with nervous system tissue in pancreas and was identified as autoantibody to glial fibrillary acidic protein (GFAP) a component of the Schwann cell mantle surrounding the pancreatic islets of Langerhans (S. R. Donev, Cell Tissue Res 237, 343-8 (1984)). These antibodies appear in female NOD-strain mice as early as 4 weeks of age and are absent in male NOD animals. Female NOD mice develop T1D at a high rate (˜90%), while male NOD mice rarely develop the disease. GFAP autoantibodies represent the first identified marker of early pre-diabetes to date, and they imply that peri-islet Schwann cells, i.e. a nervous system tissue, is an unexpected, early target of pre-diabetic autoimmunity.

Subsequent studies discovered the presence of similar autoantibodies in patients with diabetes and in relatives with high risk to develop the disease. The appearance of these autoantibodies thus provides a long elusive screening tool for the identification of early, progressive prediabetes, identifying candidates for intervention trials. Given the clear precedence of the ability of using autoantibody targets for immunotherapy (see above) (A. Atkinson, E. H. Leiter, Nat Med 5, 601-4 (1999)), it is proposed to target the autoimmune response to GFAP by immunotherapies aimed at modifying the response and halting autoimmune progression. Thus, any therapeutic modality which interferes, e.g. by interference is meant a modality having the ability to in some way alter the course, progression and/or manifestation of the disease, as a result of interfering with the disease manifestation process at the early stages focused upon by the identification of the autoimmune disease (e.g. prediabetes) indicative markers as instantly disclosed, are a part of this invention. Since the underlying autoimmunity in T1D and MS are fundamentally the same (S. Winer et al. J Immunol 166, 28-322841 (2001); S. Winer et al. J Immunol 166, 4751-4756 (2001)), it is evident that the same arguments and reasoning should apply to both diseases. Thus, it is suggested that at least several organ-selective autoimmune diseases are inherently and initially directed towards nervous system components, with disparate tissue factors and elements such as host histocompatibility molecules determining the clinical outcome. This present filing focuses on T1D and MS where relevant similarities have been worked out and reported in the literature.

Accordingly, it is an objective of the instant invention to teach a binding protein indicative of a loss of self tolerance of the Schwann cell protein, GFAP, and other SC constituents such as S-100 in mammals, notably humans (S. Schmidt et al., Brain (1997); M. Popovic, J. Sketelj, M. Bresjanac, Pflugers Arch 431, R287-8 (1996)) which will be referred to as “SC autoantibodies” and will include all immunologically detectable fragments thereof.

It is a further objective of the instant invention to teach a method and a device for the use of SC autoantibodies as a predictive marker of organ-selective autoimmune disease such as T1D and MS, either in the format of a point-of-care assay or in the format of a central laboratory diagnostic assay.

It is yet another objective of the instant invention to provide a diagnostic assay test kit for SC related autoimmune disease, notably for pre-diabetes and pre-MS.

It is a still further objective of the invention to provide a diagnostic assay test kit for prediabetes wherein the SC autoantibody is an anti-GFAP autoantibody supplied in a diagnostically effective amount and the test kit is capable of detecting binding of said diagnostically effective amount of anti-GFAP IgG with a patient sample.

It is yet another objective of the instant invention to teach therapeutic targets, therapeutic avenues and therapeutic modalities, along with methods for their determination, isolation and elucidation, which are characterized by their capability for interfering with the course, progression and/or manifestation of the disease, as a result of interfering with the disease manifestation process, for example at the early stages focused upon by the identification of the autoimmune disease (e.g. prediabetes) indicative markers as instantly disclosed.

Other objectives and advantages of this invention will become apparent from the following description taken in conjunction with the accompanying drawings wherein are set forth, by way of illustration and example, certain embodiments of this invention. The drawings constitute a part of this specification and include exemplary embodiments of the present invention and illustrate various objects and features thereof.

### BRIEF DESCRIPTION OF THE FIGURES

FIG. 1 illustrates a SELDI process using GFAP-coupled chip arrays;

FIG. 2 illustrates the presence of GFAP binding protein in 4 week old NOD female mice;

FIG. 3 illustrates a comparison of male vs. female NOD mice at 5 weeks;

FIGS. 4A-D illustrate a comparison of serum samples from patients with recent onset T1D (FIG. 4B), from auto-antibody-positive first degree relatives with probable prediabetes (FIG. 4A) and from relatives without signs of autoimmunity (FIGS. 4C and D), which were analyzed in similar fashion as NOD mice.

### DETAILED DESCRIPTION OF THE INVENTION

Since β-cells themselves express trace amounts of GAD65 as well as S-100, but lack GFAP expression detectable by RT-PCR, GFAP provides a local SC marker.

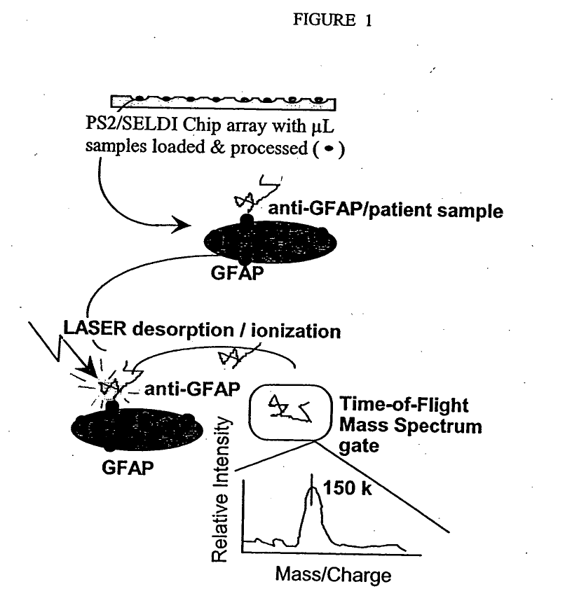

With reference to FIG. 1, IgG autoantibodies to GFAP were measured in sera from NOD mice of different ages, using covalently GFAP-coupled chip arrays in a SELDI-time-of-flight mass spectrometry instrument calibrated with a monoclonal anti-GFAP antibody.

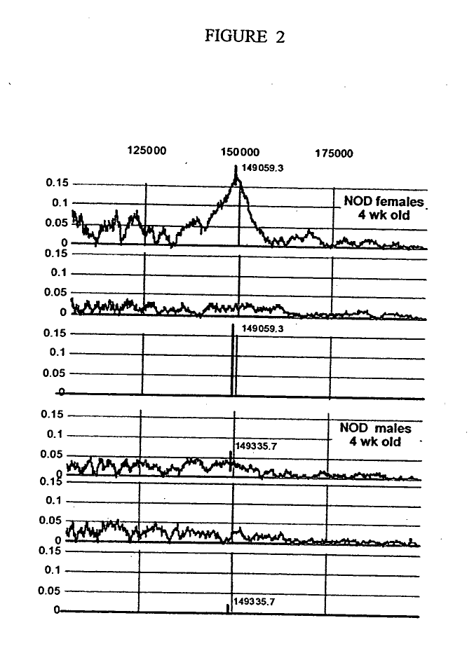

As seen in FIG. 2, serum from 11/13 NOD females as young as 4 weeks old contained a GFAP-binding protein of 149,805.71200 D mass. This 150 kD protein was removed by prior serum passage over solid phase GFAP or solid phase Protein G columns and thus represents IgG autoantibody. These autoantibodies were maintained in overtly diabetic mice 20-26 weeks of age. Samples with high autoantibody signals in SELDI-TOF-MS were found to contain anti-GFAP autoantibodies in Western blots, but the sensitivity of SELDI exceeds that of Western blots.

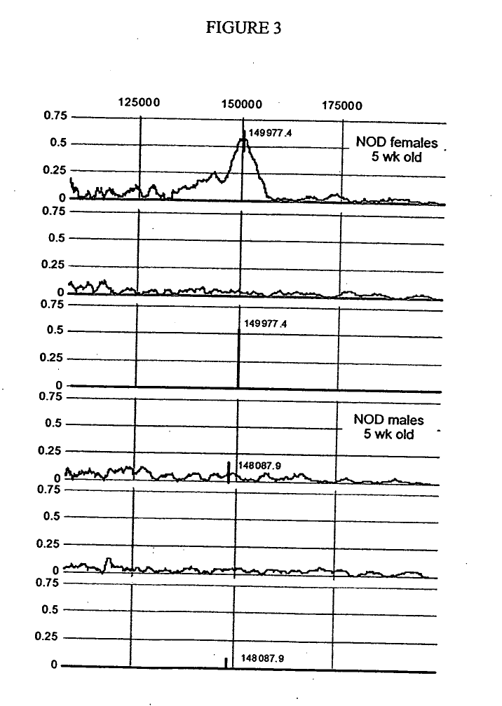

As set forth in FIG. 3, sera from male NOD mice 5-18 weeks of age, from 7 week old non-autoimmune strain C57B1/6 and 8 week old Balb/c mice, or from NOD females 3 weeks of age were negative, while 5/8 samples from 4-5 week old females were clearly positive for GFAP autoantibodies.

It was therefore concluded that loss of self-tolerance to the Schwann cell protein, GFAP, and likely other SC constituents such as S-100, is a characteristic of NOD mouse prediabetes and predicts the progressive disease course leading to overt T1D in female mice. There is no presently available serum marker to predict disease risk or overt disease in NOD mice before establishment of invasive insulits bu 10-12 weeks of age (S. Reddy, N. Bibby, R. B. Elliott, Clin Exp Immunol 81, 400-5 (1990)): in the case of NOD females GFAP autoantibodies have a positive predictive power of about 90% at an age of 5 weeks, i.e. before insulitis is established. This is an age where intervention therapies have the best effectiveness (discussed in: (S. Winer et al., J Immunol 165, 4086-4094 (2000); M. A. Atkinson, E. H. Leiter, Nat Med 5, 601-4 (1999)).

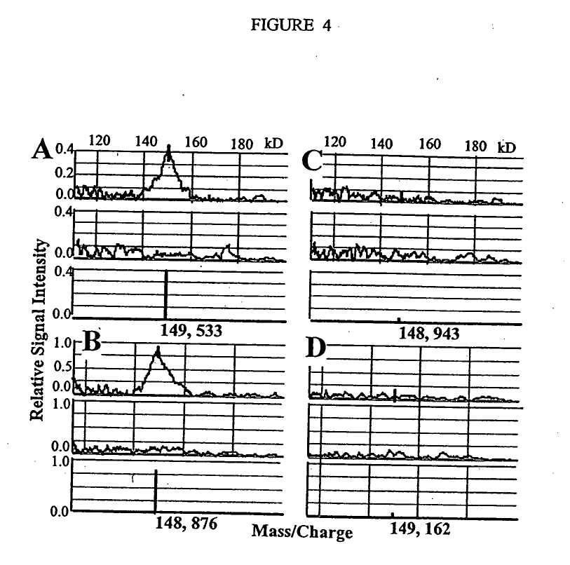

Diabetes-associated autoimmunity in NOD mice and humans targets a closely similar set of autoantigens. As seen in FIGS. 4A-D serum samples from patients with recent onset T1D (FIG. 4B), from autoantibody-positive first degree relatives with probable prediabetes (FIG. 4A) and from relatives without signs of autoimmunity (FIGS. 4C-D) were analyzed in a similar fashion as NOD mice. Samples from 24/30 new onset patients, 9/10 relatives with probable prediabetes 2/29 healthy controls, and 4/5 patients with probable MS contained anti-GFAP autoantibodies detected by SELDI-TOF-MS.

We thus conclude that autoimmunity against peri-insular SC is characteristic of human and NOD mouse T1D and probably MS and thus appears to be a characteristic of the disease in general Collectively, these observations establish pen-insular SC as a bona fide autoimmune target in T1D. Autoantibodies are not thought to be mediators of tissue destruction, but rather reflect the immune system's function to remove detritus once tissue destruction has occurred. While it is difficult to rule out subtle β-cell damage this early in the pre-diabetes process, the first autoantibody and thus the first tissue destruction in prediabetes is the peri-islet SC mantle, i.e. a nervous system tissue. This conclusion provides not only a new diagnostic element in prediabetes, but also an attractive new target for therapeutic, including immunotherapeutic intervention, e.g. modalities such as administration of an immunologically reactive moiety capable of altering the course, progression and/or manifestation of the disease, as a result of interfering with the disease manifestation process at the early stages focused upon by the identification of the disease, e.g. prediabetes indicative marker as instantly disclosed, such as by supplying a moiety capable of modifying the pathogenicity of lymphocytes specific for GFAP or other related SC components.

Therapeutic targets may thus be defined as those moieties which are capable of exerting a modulating force, wherein modulation is defined as an alteration in function inclusive of activity, synthesis, production, and circulating levels. Thus, modulation effects the level or physiological activity of at least one particular disease related biopolymer marker or any compound or biomolecule whose presence, level or activity is linked either directly or indirectly, to an alteration of the presence, level, activity or generic function of the biopolymer marker, and may include pharmaceutical agents, biomolecules that bind to the biopolymer markers, or biomolecules or complexes to which the biopolymer markers bind. The binding of the biopolymer markers and the therapeutic moiety may result in activation (agonist), inhibition (antagonist), or an increase or decrease in activity or production (modulator) of the biopolymer markers or the bound moiety. Examples of such therapeutic moieties include, but are not limited to, antibodies, oligonucleotides, proteins (e.g. receptors), RNA DNA, enzymes, peptides or small molecules.

With regard to immunotherapeutic moieties, such a moiety would be an effective analogue for a major epitope peptide in GFAP which reduces the pathogenicity of key lymphocytes which are specific for the native epitope in GFAP. An analogue is defined as having structural similarity but not identity in peptide sequencing able to be recognized by T-cells spontaneously arising and targeting the endogeneous self epitope. A critical function of this analogue is an altered T-cell activation which leads to T-cell anergy or death.

As β-cells have gene expression patterns reminiscent of neuronal cells (F. Atouf, P. Czernichow, R. Scharfmann, J Biol Chem 272, 1929-34 (1997)), it seems conceivable that interactions between peri-islet SC and intra-islet β-cells have functional interactions typical for peripheral SC and ‘their’ neurons, with the former maintaining the latter. An autoimmune attack on SC would then compromise survival of β-cells and possibly their regeneration. This possible axis of interaction has been uncovered by the observations leading to the present invention and deserve renewed attention as a candidate factor in prediabetes progression: e.g. β-cells may be victims of collateral damage in a primary autoimmune attack on pancreatic nervous system tissue.

As used herein the term “marker” or “biopolymer marker” are any molecules, typically proteins that pass out from the organ's cells as the cells become damaged or as adaptation occurs. These proteins can be either in the native form or can be any moiety which contains immunologically detectable or immunologically reactive fragments of the protein, resulting, for example, from proteolytic digestion of the protein. When the terms “marker” “biopolymer marker” or “analyte” are used, they are intended to include fragments thereof that can be immumologically detected. By “immunologically detectable” or “immunologically reactive” is meant that the protein fragments contain an epitope that is specifically recognized by a cognate antibody, e.g. the immunologically reactive marker, moiety or fragment has an affinity for a particular entity, e.g. an antibody.

As used herein, the term “antibody” includes polyclonal and monoclonal antibodies of any isotype (IgA, IgG, IgE, IgD, IgM), or an antigen-binding portion thereof, including but not limited to F(ab) and Fv fragments, single chain antibodies, chimeric antibodies, humanized antibodies, and a Fab expression library.

Antibodies useful as detector and capture antibodies in the present invention may be prepared by standard techniques well known in the art. The antibodies can be used in any type of immunoassay. This includes both the two-site sandwich assay and the single-site immunoassay of the non-competitive type, as well as in traditional competitive binding assays.

Particularly preferred, for ease and simplicity of detection, and its quantitative nature, is the sandwich or double antibody assay of which a number of variations exist, all of which are contemplated by the present invention. For example, in a typical sandwich assay, unlabeled antibody is immobilized on a solid phase, e.g. microtiter plate, and the sample to be tested is added. After a certain period of incubation to allow formation of an antibody-antigen complex, a second antibody, labeled with a reporter molecule capable of inducing a detectable signal, is added and incubation is continued to allow sufficient time for binding with the antigen at a different site, resulting with a formation of a complex of antibody-antigen-labeled antibody. The presence of the antigen is determined by observation of a signal which may be quantitated by comparison with control samples containing known amounts of antigen.

The assays may be competitive assays, sandwich assays, and the label may be selected from the group of well-known labels such as radioimmunoassay, fluorescent or chemiluminescence immunoassay, or immunoPCR technology. Extensive discussion of the known immunoassay techniques is not required here since these are known to those of skill in the art. See Takahashi et al. (Clinical Chemistry 45(8):1307 1999) for S-100β assay.

Although not wishing to be limited to any particular embodiment, the panel format exemplified herein is known and commercially available. The panel format is similar to a format currently being used in association with pregnancy testing and is commercially available under the trademark BIOSIGN. Any assay device or method in accordance with the objectives of the instant invention is contemplated for use with one or more bodily fluids, said bodily fluids being selected from the group consisting of blood, blood components, urine, saliva, lymph and cerebrospinal fluid.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement of parts herein described and shown. It will be apparent to those skilled in the art that various changes may be made without departing from the scope of the invention and the invention is not to be considered limited to what is shown and described in the specification and drawings. One skilled in the art will readily appreciate that the present invention is well adapted to carry out the objectives and obtain the ends and advantages mentioned, as well as those inherent therein. The various biomolecules, e.g. antibodies, markers, oligonucleotides, peptides, polypeptides, biologically related compounds, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1. A method for diagnosing pre-Type 1 diabetes comprising the steps of:
(a) obtaining a sample of a bodily fluid from a non-diabetic patient, and;
(b) analyzing said sample for the presence of at least one Schwann cell autoantibody or an immunologically detectable fragment thereof wherein the presence of said at least one Schwann cell autoantibody or an immunologically detectable fragment thereof is diagnostic for pre-Type 1 diabetes.

2. The method according to claim 1 wherein said Schwann cell autoantibody or immunologically detectable fragment thereof is auto-reactive with glial fibrillary acidic protein (GFAP).

3. The method according to claim 1 wherein said sample of a bodily fluid is selected from the group consisting of blood, blood products, urine, saliva, cerebrospinal fluid, and lymph.
